(19) United States
(12) Patent Application Publication
(10) Pub. No.: US 2014/0012295 A1
(43) Pub. Date: Jan. 9, 2014

(54) BLADE FOR PREPARING AN ENDOTHELIAL GRAFT, AND ASSOCIATED PREPARATION METHOD

(71) Applicant: CENTRE HOSPITALIER UNIVERSITAIRE DE ROUEN, (US)

(72) Inventor: Marc MURAINE, BIHOREL (FR)

(73) Assignee: CENTRE HOSPITALIER UNIVERSITAIRE DE ROUEN, ROUEN cedex (FR)

(21) Appl. No.: 13/714,063

(22) Filed: Dec. 13, 2012

(30) Foreign Application Priority Data
Jul. 6, 2012 (FR) 12 56559

(51) Int. Cl. A61F 9/013

(52) U.S. Cl. CPC A61F 9/0133
USPC 606/166

(57) ABSTRACT

The invention relates to an instrument for preparing an corneal endothelial graft, the tool being a trephine blade (1) having a cross section in the form of an arc of a circle of between 270° and 355°. The invention also relates to a method of preparing a graft using such an instrument.

### FIELD OF THE INVENTION

The invention relates to a method for the preparation of corneal endothelial grafts, and instruments suitable for the said preparation.

### PRIOR ART

Significant advances have been made in endothelial grafting techniques over the past ten years and these have progressively replaced penetrating keratoplasty when a corneal graft is required as a result of Fuchs' corneal dystrophy, bullous keratopathy, or the failure of an earlier penetrating keratoplasty. These new techniques, in which only the diseased portion of the cornea is replaced, have proven to be superior to replacing the entire cornea. Inserting the endothelial graft through a small incision makes it possible to reduce astigmatism, avoid the weakening that comes with a circular trephination, and speed visual recovery.

At the present time there are two main techniques for preparing the endothelial graft. The most popular is known as DSAEK (Descemet's Stripping Automated Endothelial Keratoplasty). In this technique, the posterior graft is created using a microkeratome, or a femtosecond laser. This technique has the advantage of simplicity, both when cutting the graft and when introducing it into the anterior chamber. However the graft increases overall thickness, because it includes some residual stroma.

The second technique is DMEK (Descemet Membrane Endothelial Keratoplasty), in which the posterior graft comprises only the Descemet membrane and the endothelium. In this case, the graft is prepared manually.

Although no randomized study has been carried out to compare the two techniques, it is evident, from reading articles reporting on the results following endothelial grafting, or retrospective comparative studies comparing them, that DMEK offers patients better visual recovery due to the absence of residual stroma.

However, the DMEK techniques is difficult to perform, and there is always a substantial risk of tearing the endothelium during the graft preparation.

### SUMMARY OF THE INVENTION

It is an objective of the invention to alleviate the abovementioned disadvantage by proposing an instrument and a method for preparing a Descemet graft that presents a lower risk of tearing the endothelium.

The second objective of the invention is to allow preparation of a pure Descemet graft that is easier than the technique hitherto described.

To these ends, the invention proposes a hollow cylindrical blade capable of cutting the Descemet's membrane and the endothelium of a cornea. The blade is further characterized by having a cross section in the form of an arc of a circle extending over between 270° and 355°

The blade according to a preferred embodiment of the invention also has at least one of the following features:
the arc of a circle has two ends bent over towards the outside of the circle.
the blade has a cross section of a diameter of 6 to 9 mm.
the arc of a circle is 320°. Inbetween 270° and 355°

The invention also proposes a method for preparing an endothelial graft, involving trephining the donor cornea using a blade according to the invention, said trephination extending over an arc of a circle of between 345 and 355°.

### BRIEF DESCRIPTION OF THE FIGURES

Other features, objects and advantages of the present invention will become apparent from reading the following description, with reference to the attached figures, which are given as non limiting examples, and in which:

FIG. 1 depicts the preparation of an endothelial graft.

FIGS. 2a and 2b respectively are a cross sectional view and a perspective view of a blade according to the invention.

### DESCRIPTION OF ONE EMBODIMENT OF THE INVENTION

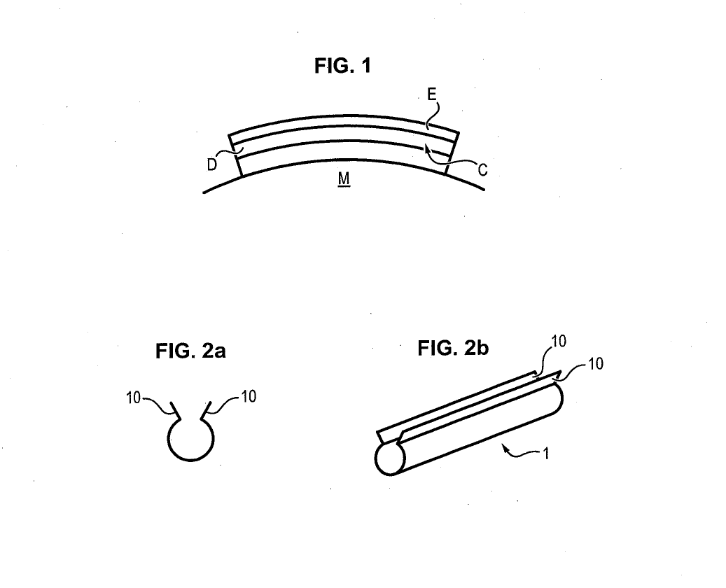

Reference is made to FIG. 1 which depicts the preparation of a graft for an endothelial grafting procedure. The cornea C is positioned on an artificial anterior chamber (M), with the endothelium E uppermost. The cornea rests in a stable position on the concave part of this anterior chamber. It is to be noted that the graft could also be placed first on a punch block and after threphination, placed in the artificial chamber.

Reference is made to FIGS. 2a and 2b which depict a blade 1 suited to the preparation of the graft. This blade is of the trephine type, i.eit has a cylindrical shape of hollow circular cross section. It preferably has a diameter of 8 mm. In use, the blade is pressed firmly against the corneal endothelium so as to cut through the Descemet membrane D in a circular manner.

According to the invention, the blade 1 does not have a closed circular cross section but has a cross section that forms an arc of a circle between 270° and 355° preferably 320°.

Advantageously, the ends 10 of the arc of a circle formed by the blade section are bent outwards (the inside being considered to be toward the center of the circle formed by the blade section). This allows the trephination blade 1 to align with the artificial anterior chamber.

The artificial anterior chamber is then closed again, with the endothelium still uppermost, and air is introduced into the chamber. The cornea is then inverted, with the endothelium bulging upwards. A microsponge is used to remove the surplus culture medium from the side. The endothelium is stained using Trypan blue so that the Descemet trephination region can be seen clearly.

By using the trephination blade 1 according to the invention, there is, in the trephination area, a contiguous region between the central endothelium and the peripheral endothelium. On each side of this region, the peripheral endothelium can be removed very simply in a single operation using Troutmann forceps. The endothelium is then torn at the periphery opposite the contiguous region and in such a way as to create a small flap that is easy to lift. A small spatula can then be slid under this flap, guiding it towards the center of the cornea, but only over a length of 3 mm. Once this has been done, the rest of the dissection can be performed using a disposable 27-gauge cannula fitted to a 2.5 ml syringe filled with culture medium or balanced salt solution.

During the detachment procedure, the endothelium is covered with visco elastic.

The 27-gauge cannula is slid under the endothelium towards the center of the cornea. As culture medium is injected, the endothelium will gradually become detached by hydrodissection at the front of the cannula. Once progress has reached the center of the endothelium, the Descemet membrane can very easily be separated on either side, from right to left, up to the trephination region. Hydrodissection can then be continued as far as the opposite Descemet trephination region whereupon the endothelium can be detached on either side as far as the periphery. This then yields a pure Descemet graft 8 mm in diameter, laying flat on the donor cornea with the endothelium facing upwards.

For insertion in the recipient eye, the graft is covered with visco elastic then folded, endothelium inwards.

The Descemet graft is rolled up, with the endothelium on the inside, in a visco jet implant injection cartridge intended for implants.

Inside the cartridge, an extended Sinskey hook is used to guide the rolled-up graft to the injection barrel and pull out so that it positions itself near the orifice. Once the diseased endothelium has been removed from the patient through a 2.8 mm incision, the cartridge is fixed at the end of a cannula suited to the internal diameter of the cartridge and attached to a 2.5 ml syringe containing Balanced Salt solution. The cartridge is then inserted into the incision, and the endothelial graft is inserted into the anterior chamber via the injection of Balanced Salt solution. The gradual unrolling of the graft occurs as a result of the injection of BSS and may take a certain length of time. Once, the centration appears to be correct, an air bubble is used to hold the graft at the center of the cornea.

Deploying the graft, endothelium downward, is easier than in techniques in which the endothelium is initially directed toward the cornea. All techniques however, may require a certain degree of manipulation and surgical skill.

At the end of the intervention, the graft is held in place by the air bubble, kept in the eye for 24 hours.

1. A hollow cylindrical blade capable of cutting Descemet membrane and endothelium of a cornea, wherein the blade has a cross section in the form of an arc of a circle extending over between 270° and 355°.

2. The blade according to claim 1, in which the arc of a circle has two ends bent over towards the outside of the circle.

3. The blade according to claim 1, having a cross section of a diameter of 6 to 9 mm.

4. The blade according to claim 1, in which the arc of a circle extends over 320°.

5. The blade according to claim 1, in which the blade is directly used on the artificial chamber in which the cornea C is positioned and pressurized with the endothelium E uppermost.

6. A method for preparing an endothelial graft, comprising trephining a cornea using blade according to claim 1, said trephination extending over an arc of a circle of between 270° and 355°.
